(19) United States
(12) Patent Application Publication
(10) Pub. No.: US 2025/0195623 A1
(43) Pub. Date: Jun. 19, 2025

(54) METHOD FOR PRODUCING A STABILIZED DIAMINE OXIDASE LYOPHILISATE AND PHARMACEUTICAL DOSAGE FORM

(71) Applicant: STADA ARZNEIMITTEL AG, Bad Vilbel (DE)

(72) Inventors: Florian FORSTER, Zeiselmauer (AT); Sandra GIRSTMAIR, Tulln (AT); Angela LINDER, Unteroberndorf (AT)

(21) Appl. No.: 18/985,467

(22) Filed: Dec. 18, 2024

(30) Foreign Application Priority Data
Dec. 19, 2023 (EP) 23218109.9

(51) Int. Cl. A61K 38/44; A61K 9/19; A61K 47/18; A61K 47/26; C12N 9/06; C12N 9/96

(52) U.S. Cl. CPC A61K 38/44; A61K 9/19; A61K 47/183; A61K 47/26; C12N 9/0022; C12N 9/96; C12Y 104/03022

(57) ABSTRACT

Disclosed is a method for producing a stabilized lyophilisate with plant-based diamine oxidase (DAO), comprising the following steps: Providing DAO-containing plants or DAO-containing parts thereof; Homogenizing the DAO-containing plants or DAO-containing parts thereof in aqueous solution to obtain an aqueous homogenate; Adding (a) a sugar or sugar alcohol and (b) an amino acid as a stabilizer; and Lyophilizing the aqueous homogenate or a DAO-containing fraction thereof in the presence of the stabilizer. Also disclosed is a lyophilisate obtainable by this method, as well as a solid pharmaceutical dosage form for oral administration containing this lyophilisate. This dosage form is intended for therapeutic use, preferably in the treatment of histamine-induced disorders, or can be used as a dietary supplement or dietary food.

### CROSS-REFERENCE TO RELATED APPLICATIONS

This application claims priority to European Application No. 23218109.9, filed Dec. 19, 2023, the contents of which is incorporated into the present application by reference.

### BACKGROUND

The present invention relates to a method for producing a stabilized lyophilisate with plant-based diamine oxidase (DAO), and to a stabilized lyophilisate with plant-based DAO.

Histamine (2-(4-imidazolyl)ethylamine) is a biogenic amine that is involved in many physiological processes in the organism, but is also found in some foods. Histamine can trigger various, often nonspecific symptoms that are often similar to allergic reactions in people who suffer from a histamine intolerance.

High concentrations of free circulating histamine can, among other things, trigger symptoms such as headache, stuffed or runny nose, airway obstructions, tachycardia and extrasystoles, as well as stomach and intestinal complaints, which can lead to soft stools to diarrhea, and hypotension. Often swelling of the eyelids, occasionally also urticarial rashes are described.

In addition, skin redness, drop in blood pressure and bronchospasm may occur.

DAO (EC 1.4.3.22) is an enzyme that occurs in various plants, animals and microorganisms and is responsible for the degradation of biogenic amines, in particular histamine. DAO is also known as histaminase.

Various DAO-containing preparations are known in the prior art, which can be ingested with meals by persons suffering from histamine intolerances in order to support the breakdown of histamine from the food in the intestine and thereby reduce or completely prevent the undesirable symptoms caused by the histamine ingested through the food.

Thus, WO 2006/003213 A1 discloses pharmaceutical compositions, dietary supplements, and cosmetic compositions comprising DAO and uses thereof. In particular, the DAO is formulated to be enteric. Animal sources for the DAO are preferred, in particular DAO from porcine kidneys.

However, in recent years, DAO preparations based on plants have also gained in importance.

Among other things, the general acceptance of herbal medicines and dietary supplements is higher, especially among people with ethical or religious concerns about the use of animal products. In addition, extracting DAO from sustainably grown plants can be more environmentally and climate-friendly than extracting it from animal sources.

Thus, US 2014/004179 A1 relates to a composition with DAO for use in the treatment or prevention of fibromyalgia or chronic fatigue syndrome. The DAO can also come from plant sources.

WO 02/43745 A2 relates to a histaminase of plant origin for use in the treatment of allergic and septic shock as well as allergic asthma.

Proteins and in particular enzymes are susceptible to physical and chemical degradation processes and are therefore fundamentally unstable over longer storage times. This also applies to plant-based DAO, where the instability of the enzyme DAO is manifested in the loss of enzymatic activity, which increases over time.

The production of pharmaceutical dosage forms and dietary supplements containing plant-based DAO consequently requires a suitable stabilization step. A common method for stabilizing enzymes and other biological materials is lyophilization or freeze-drying. This method comprises freezing the enzyme-containing solutions, followed by removing water via sublimation under vacuum at low temperatures.

However, lyophilization can also contribute to the instability of proteins and enzymes, as physical stress is exerted on the proteins due to the phase transitions. In addition, excipients that serve to stabilize the protein structure during lyophilization, and are therefore added before lyophilization, do not necessarily also ensure the stability of the proteins during long-term storage (as a lyophilisate), and can sometimes even impair them.

The most common mechanisms that lead to protein degradation in the long term are hydrolysis, oxidation and deamination. As countermeasures, stabilizers are often added to protect the proteins. The choice of these stabilizers has a major impact on the long-term stability of proteins. It also depends on which protein or enzyme is to be stabilized, as these do not necessarily behave the same during lyophilization or subsequent storage.

Padma et al. (Process Biochemistry 43.10 (2008): 1019-1032) is a review article on enzyme stabilization in aqueous and non-aqueous environments.

Ó'Fagáin (Enzyme and Microbial Technology 33.2-3 (2003): 137-149) is another review article on enzyme stabilization.

Forney-Stevens, et al. (Journal of Pharmaceutical Sciences 105.2 (2016): 697-704) is an article investigating the use of amino acids as excipients to improve the stability of sucrose-based lyophilized protein formulations.

Alemany-Fornes, et al. (bioRxiv (2023): 2023-04) is a preprint in which the DAO activity of various Over-The-Counter (OTC)-preparations available on the market is investigated.

In order to ensure the long-term stability of the plant-based DAO, suitable stabilizers must be used that offer the best possible protection of the plant-based DAO both during the lyophilization process and during long-term storage.

Megoura et al. (Molecules 28 (2023): 992) deals with the improvement of the stability of DAO from peas (Pisum sativum) by trehalose or sucrose. The DAO was lyophilized in the absence or presence of sucrose or trehalose and the stability of the resulting preparations was monitored during storage at 4° C. and −20° C. for 18 months. Tablets were also formulated by direct compression from the lyophilisates stored at −20° C. The tablets were stored at 4° C. and stability tracked over 6 months. The DAO powders freeze-dried with sucrose or trehalose stabilizer or the tablets produced from them had a higher enzyme activity after storage compared to DAO powders or tablets that did not contain sucrose or trehalose.

In this study, however, no stability studies were carried out at room temperature (25° C. or higher, for example 30° C.). However, these storage conditions are of high practical relevance, since the logistical effort involved in the continuous cooling of medicinal products or food supplements is associated with high costs and is therefore generally unacceptable. In addition, due to non-linear effects on protein stability, quantitative conclusions from the observations made at significantly lower temperatures on the effects of stabilizers at room temperature are not permissible in all cases. Due to the complex interaction between the individual possible stabilizers and the enzyme DAO, it is difficult to predict what effect an excipient or a class of excipients will have on the stability of the protein. There is therefore still a need for further optimisation of the excipients for stabilising plant-based DAO during and after lyophilization, in particular during subsequent storage at room temperature.

### SUMMARY

It is therefore an object of the present invention to provide a method for producing a stabilized lyophilisate with plant-based DAO and a corresponding stabilized lyophilisate, wherein the lyophilisate should remain as stable as possible, i.e. should have as little loss of activity as possible, even under conditions relevant to practice (such as storage at room temperature and/or increased humidity).

### BRIEF DESCRIPTION OF THE DRAWINGS

FIG. 1: Residual activity of the DAO in a lyophilized pea sprout homogenate stored at 30° C., 65% relative humidity in an open container mixed with the additives (each in a weight ratio of additive to homogenate of 1:1): arginine monohydrochloride (“arginine”), trehalose (“trehalose”), as well as the calculated residual activity of the lyophilized pea sprout homogenate with the addition of arginine monohydrochloride and trehalose in combination (“trehalose+arginine calculated”). In comparison, the expected residual activity of the combination of arginine monohydrochloride and trehalose predicted from the DoE model (“Trehalose+Arginine DoE model prediction”). The error indicators indicate the 95% confidence interval. There was an effect of arginine and trehalose that was higher than expected.

FIG. 2: Stability study of the pea sprout homogenate formulations derived from the DoE model with arginine monohydrochloride in a weight ratio of arginine monohydrochloride to homogenate of 1:1 (“100% arginine”), trehalose (in a weight ratio of trehalose to homogenate of 1:2) and arginine monohydrochloride in a weight ratio of 1:1 to homogenate (“50% trehalose, 100% arginine”) as well as trehalose (in a weight ratio of trehalose to homogenate of 1:1) and arginine monohydrochloride in a weight ratio of 1:1 to homogenate (“100% trehalose, 100% arginine”). The data shows the monthly progression of the DAO activity decrease within a storage period of 6 months (“1M”, “2M”, “3M”, “4M”, “5M”, “6M”) with reference to the starting value (“Start”) as 100%. (a) Storage at 25° C., 60% RH, (b) Storage at 30° C., 65% RH.

FIG. 3: Synergistic effect when using trehalose and arginine (in the form of arginine hydrochloride—“arginine*HCl”) together. The progression of the DAO activity decrease at different points in time is shown, with reference to the starting value (“homogenate before lyo”) as 100%. In the last group (“trehalose and arginine*HCl after lyo”), trehalose and arginine were added only after lyophilization. “Lyo”—lyophilization.

FIG. 4: Addition of additives in different amounts relative to the total protein, expressed as the ratio of total protein:trehalose:arginine*HCl (on a weight basis). The progression of the DAO activity decrease at different points in time is shown, with reference to the starting value as 100%. “Arginine*HCl”—arginine hydrochloride.

### DETAILED DESCRIPTION OF THE INVENTION

In a first aspect, the present invention provides a method for producing a stabilized plant-based DAO lyophilisate comprising the steps:
Providing DAO-containing plants or DAO-containing parts thereof (in particular plant sprouts);
Homogenizing the DAO-containing plants or DAO-containing parts thereof in aqueous solution to obtain an aqueous homogenate;
Adding (a) a sugar or sugar alcohol and (b) an amino acid (these preferably serve as stabilizers); and
Lyophilizing the aqueous homogenate or a DAO-containing fraction thereof in the presence of (a) the sugar or sugar alcohol and (b) the amino acid.

In addition, the present invention provides a stabilized plant-based DAO lyophilisate obtainable by this method.

In another aspect, the present invention provides a stabilized plant-based DAO lyophilisate comprising (a) a sugar or sugar alcohol and (b) a free amino acid. These two components preferably serve as stabilizers (in other words, components (a) and (b) are preferably present as stabilizers).

A free amino acid is an amino acid that is not part of a peptide or protein.

Furthermore, a solid pharmaceutical dosage form (in particular a capsule or tablet) is provided which contains one of the abovementioned lyophilisates according to the invention, optionally in compressed form. This dosage form can be used for therapeutic use, preferably in the treatment of histamine-induced disorders, or used as a dietary supplement or dietary food. Surprisingly, it has been found that combinations of a sugar or sugar alcohol with an amino acid (in particular those specifically disclosed below) are particularly suitable for stabilizing the plant-based DAO, in particular with regard to long-term storage, and in particular at room temperature (and a certain humidity). It has been found that the stabilization effect as a result of the presence of these two components is additive or even synergistic.

In a preferred embodiment of the method according to the invention, the amino acid is a free amino acid.

In a preferred embodiment of the method according to the invention or of the lyophilisate according to the invention, the amino acid or the free amino acid is preferably selected from the group consisting of basic amino acids such as arginine, histidine and lysine as well as the amino acids asparagine, glutamine, cysteine, homocysteine, methionine, tryptophan and tyrosine. Particular preference is given to basic amino acids, in particular arginine, histidine and lysine, preferably arginine.

It goes without saying that the free amino acid can also be used according to the invention in the form of a pharmaceutically acceptable salt. In the case of arginine, this is arginine hydrochloride, for example.

Consequently, in a further preferred embodiment (before addition), the amino acid is present in a pharmaceutically acceptable salt form, preferably as a hydrochloride, sulfate, acetate, phosphate, tartrate or citrate salt, in particular arginine hydrochloride, arginine sulfate, arginine acetate, histidine hydrochloride, histidine sulfate, histidine acetate, lysine hydrochloride, lysine sulfate or lysine acetate. For example, an aqueous solution of the salt form used for addition may be prepared in advance. Arginine hydrochloride is particularly preferred.

A further preferred embodiment of the method according to the invention or of the stabilized lyophilisate according to the invention provides that the sugar or sugar alcohol is selected from the group consisting of disaccharides such as trehalose, sucrose, lactose and maltose, trisaccharides such as raffinose, polysaccharides such as maltodextrin and the sugar alcohols mannitol, sorbitol, glycerol, xylitol, erythritol and inositol. In a particularly preferred embodiment, the sugar is a disaccharide, in particular trehalose.

In a further preferred embodiment of the method according to the invention, (a) the sugar or sugar alcohol is preferably added as a stabilizer in an amount of at least 10% (w/w) of the homogenate, more preferably at least 20% (w/w), even more preferably at least 30% (w/w), in particular at least 40% (w/w) or even at least 50% (w/w) of the homogenate, and/or (b) the amino acid or the free amino acid is preferably added as a stabilizer in an amount of at least 20% (w/w) of the homogenate, more preferably at least 40% (w/w), even more preferably at least 60% (w/w), in particular at least 80% (w/w) or even at least 100% (w/w) of the homogenate (on a weight basis).

In a further preferred embodiment of the stabilized lyophilisate with plant-based DAO according to the invention, the lyophilisate comprises (a) a sugar or sugar alcohol in a weight ratio of at least 1:1 or even at least 2:1, preferably at least 4:1 or even at least 7:1, preferably at least 10:1 or even at least 13:1, more preferably at least 16:1 or even at least 19:1, in particular at least 22:1 or even at least 25:1 to the total protein content of the lyophilisate, and/or (b) a free amino acid in a weight ratio of at least 1:1, preferably at least 4:1 or even at least 7:1, preferably at least 10:1 or even at least 18:1, more preferably at least 25:1 or even at least 32:1, in particular at least 40:1 or even at least 50:1 to the total protein content of the lyophilisate.

In other words, it is preferable for both the sugar or sugar alcohol and the amino acid to be present in excess of the total protein content on a weight basis.

In a further preferred embodiment of the method according to the invention or of the stabilized lyophilisate according to the invention, the DAO-containing plants originate from the Leguminosae family, preferably being selected from the genera Pisum, Lens, Cicer, Lathyrus, Phaseolus and Vicia. These plants (especially their plant sprouts) are good sources of a plant-based DAO that can be stabilized according to the present invention. Particular preference is given to peas (Pisum sativum), lentils (Lens culinaris), chickpeas (Cicer arietinum), seed peas (Lathyrus sativus), beans (Phaseolus vulgaris) and field beans (Vicia faba), in particular peas (Pisum sativum).

Plant sprouts are the early growth stage of plants that begins shortly after seed germination.

At this stage, plants have a high concentration of nutrients, vitamins, minerals and enzymes (including DAO), as they are needed for the rapid growth and development of the young plant. Therefore, in a preferred embodiment, the DAO-containing plants are sprouts.

In a further preferred embodiment of the method according to the invention or of the stabilized lyophilisate according to the invention, the DAO-containing plants are seedlings.

In a further preferred embodiment of the method according to the invention or of the stabilized lyophilisate according to the invention, at least one further stabilizer or excipient is added. The addition of these additional stabilizers can further improve the stability of the lyophilisate while reducing possible harmful reactions or oxidation processes that could adversely affect the quality of the final product. Other excipients, in turn, can perform a variety of functions, such as protecting the protein during freezing or during the drying process during the lyophilization process or improving the lyophilizability or reducing aggregation in the lyophilisate (if it is in ground form). Antioxidants such as lecithin, tocopherol, resveratrol, lactic acid, glutathione and/or ascorbic acid and/or cryoprotectors are particularly preferred as additional stabilizers. The additional stabilizers and/or excipients may be added before or after lyophilization. If the stabilizer is a cryoprotector, it is added before lyophilization.

According to a further preferred embodiment of the method according to the invention, the production process of the stabilized lyophilisate additionally comprises grinding after the lyophilization. Grinding can be used in particular if the lyophilisate is intended for further processing in capsules and tablets. The finer particle size facilitates processing and allows uniform distribution of the active ingredient in the solid dosage form. This leads to a more consistent release of the active substance in the gastrointestinal tract and thus to improved bioavailability.

After grinding, the lyophilisate can be filled into capsules or used for the production of tablets, pellets, micropellets, granules or jelly beans. In the production of these dosage forms, the ground lyophilisate can, for example, be mixed with other excipients, such as binders, flow regulators or lubricants, and then optionally pressed to obtain the desired size and shape.

The solid (pharmaceutical) dosage form according to the invention can comprise one or more (pharmaceutically acceptable) excipients, for example a substance selected from the group of polysaccharides, such as cellulose (e.g. microcrystalline cellulose), hydroxypropylmethylcellulose, carboxymethylcellulose, hydroxyethylcellulose, ethylhydroxyethylcellulose, hydroxypropylcellulose, cellulose acetyl phthalate, hydroxypropylmethylcellulose phthalate, cellulose acetate, cellulose propionate, cellulose acetate butyrate, ethylcellulose, guar flour, alginic acid and/or alginates, pectin, polyvinylpyrrolidone, polyvinyl acetate, polyvinyl alcohol, polyvinyl chloride, polymers of acrylic acid and their salts or polyacrylates or polymethacrylates or co-polymers thereof (e.g. Eudragit® E, Eudragit® R, Eudragit® S, Eudragit® NE, Eudragit® RS, Eudragit® RL), Vinylpyrrolidone-vinyl acetate copolymers, shellac, nylon, colloidal silica (e.g. Aerosil®), behenates (e.g. glyceryl dibehenate), stearates, polyamide, polyacrylamide, polyethylene, polyalkylene glycols such as polyethylene glycol, copolymers of polyalkylene glycols, e.g. of polyethylene glycol and polypropylene glycol (Pluronic®, BASF); and mixtures thereof. In particular, if the dosage form is a tablet, it may contain, for example, a filler such as lactose, a disintegrant such as microcrystalline cellulose, a flow agent such as colloidal silica and/or a lubricant such as magnesium stearate as excipients.

According to a preferred embodiment, the dosage form according to the invention is enteric in order to enable a targeted release of the DAO in the intestine. Enteric coating can be achieved, for example, with coatings (e.g. tablets, capsules or pellets), which are only dissolved at a higher pH value, such as is present in the intestine. This ensures that the sensitive DAO present in the dosage form passes through the stomach undamaged. Examples of suitable enteric coatings include polymers such as polyvinyl acetate phthalate (PVAP), hydroxypropyl methyl cellulose phthalate (HPMCP), cellulose acetate phthalate (CAP), cellulose acetate succinate, hydroxypropyl methyl cellulose acetate succinate, or shellac. It is also possible to use enteric coatings based on methacrylic acid copolymers, such as methyl acrylate-methacrylic acid copolymers or methyl methacrylate-methacrylic acid copolymers.

Another preferred polymer for an enteric coating of the dosage form according to the invention is ethylcellulose, preferably in combination with alginate (e.g. based on Surelease®, Colorcon Ltd., United Kingdom). The dosage form can be present, for example, in an enteric form, as disclosed, for example, in WO 2006/003213 A1.

In a further preferred embodiment, the dosage form according to the invention is a capsule, a tablet or a sachet. For example, if the dosage form is a capsule, its shell may consist essentially of hard gelatin.

In a further embodiment, the dosage form according to the invention is a sustained-release dosage form (i.e. a dosage form with a delayed release) such as a sustained-release tablet.

How a dosage form can be formulated in a sustained-release form is known in the prior art, for example from WO 2010/028794 A1. In the embodiment according to the invention, a polymer is preferably used as a retarding excipient, the polymer being selected from the neutral homopolymers and copolymers of (meth)acrylic acid esters, cationic homopolymers and copolymers of (meth)acrylic acid esters with quaternary ammonium groups, polyvinyl acetate, cellulose acetate, cellulose propionate, cellulose acetate butyrate and ethylcellulose.

As already mentioned at the beginning, dietary, exogenous histamine can also trigger a variety of disorders due to unwanted reactions, including headaches, rhinitis, tachycardia, stomach and intestinal complaints and hypotension. The invention further relates to the solid dosage form according to the invention containing the lyophilisate according to the invention for therapeutic use, in particular for use in the treatment of histamine-induced diseases or disorders. The DAO activity (expressed in kilo histamine degrading units (kHDU)/g of lyophilisate) can be determined, for example, according to the following measurement method: DAO REA (“DAO specific radioextraction assay”). Particular preference is given to a DAO activity in the lyophilisate according to the invention of at least 5000 kHDU/g, preferably at least 10,000 kHDU/g, even more preferably at least 12500 kHDU/g, in particular at least 15,000 kHDU/g or even at least 25,000 kHDU/g. The activity in the lyophilisate according to the invention is usually below 100,000 kHDU/g, in particular below 80,000 kHDU/g or below 70,000 kHDU/g.

The DAO REA is disclosed, for example, in Mayer et al. (Allergologie 28.1 (2005): 1-8). The DAO REA is preferably carried out as disclosed in this publication, the unit being convertible into HDU as specified in the next paragraph.

The measurement of DAO activity by means of DAO REA and indication in HDU is also used in the Implementing Regulation (EU) 2023/951 of the 12 May 2023 commission for amending the Implementing Regulation (EU) 2017/2470 as regards the specifications of the novel food protein extract from porcine kidneys. As can be seen from these implementing regulations, 48,000 HDU corresponds to 1 mU for the DAO REA.

The total protein content in the lyophilisate according to the invention can be determined by calculating the drying yield and determining the protein content in the pea sprout homogenate using known methods, such as, for example, by means of the BCA test (bicinchonic acid) (Smith, P. K. et al., Measurement of protein using bicinchoninic acid, Analytical Biochemistry 150(1): 76-85, 1985), the Dumas method (DIN EN ISO 16634-1:2009-07) or the Kjeldahl method.

The invention further relates to the following embodiments:
Embodiment 1. A method for producing a stabilized lyophilisate with plant-based diamine oxidase (DAO), comprising the following steps:
   Providing DAO-containing plants or DAO-containing parts thereof;
   Homogenizing the DAO-containing plants or DAO-containing parts thereof in aqueous solution to obtain an aqueous homogenate;
   Adding (a) a sugar or sugar alcohol and (b) an amino acid; and
   Lyophilizing the aqueous homogenate or a DAO-containing fraction thereof in the presence of (a) the sugar or sugar alcohol and (b) the amino acid.
Embodiment 2. The method according to embodiment 1, wherein the amino acid is a free amino acid.
Embodiment 3. The method according to embodiment 1 or 2, wherein the amino acid is selected from the group consisting of basic amino acids such as arginine, histidine, and lysine, and the amino acids asparagine, glutamine, cysteine, homocysteine, methionine, tryptophan, and tyrosine.
Embodiment 4. The method according to embodiment 3, wherein the amino acid is a basic amino acid.
Embodiment 5. The method according to embodiment 4, wherein the amino acid is arginine.
Embodiment 6. The method according to any one of embodiments 1 to 5, wherein the sugar or sugar alcohol is selected from the group consisting of disaccharides such as trehalose, sucrose, lactose, and maltose, trisaccharides such as raffinose, polysaccharides such as maltodextrin, and the sugar alcohols mannitol, sorbitol, glycerol, xylitol, erythritol, and inositol.
Embodiment 7. The method according to embodiment 6, wherein the sugar or sugar alcohol is a disaccharide.
Embodiment 8. The method according to embodiment 7, wherein the sugar or sugar alcohol is trehalose.
Embodiment 9. The method according to any one of embodiments 1 to 8, wherein the DAO-containing plants are members of the Leguminosae family, preferably selected from the genera Pisum, Lens, Cicer, Lathyrus, Phaseolus, and Vicia.
Embodiment 10. The method according to any one of embodiments 1 to 9, wherein the DAO containing plants are selected from peas (Pisum sativum), lentils (Lens culinaris), chickpea (Cicer arietinum), seed beans (Lathyrus sativus), beans (Phaseolus vulgaris), and field beans (Vicia faba).
Embodiment 11. The method according to embodiment 10, wherein the DAO-containing plants are peas (Pisum sativum).
Embodiment 12. The method according to any of embodiments 1 to 11, wherein the DAO-containing plants are sprouts or seedlings.
Embodiment 13. The method according to any of embodiments 1 to 12, wherein the sugar or sugar alcohol is added in an amount of at least 10% (w/w) of the homogenate, more preferably at least 20% (w/w), even more preferably at least 30% (w/w), in particular at least 40% (w/w) or even at least 50% (w/w) as a stabilizer.
Embodiment 14. The method according to any of embodiments 1 to 13, wherein the amino acid is added as a stabilizer in an amount of at least 20% (w/w) of the homogenate, more preferably at least 40% (w/w), even more preferably at least 60% (w/w), in particular at least 80% (w/w) or even at least 100% (w/w) of the homogenate.
Embodiment 15. The method according to any one of embodiments 1 to 14, wherein at least one further stabilizer such as an antioxidant or a cryoprotector or an excipient is added, in particular glutathione, lecithin, tocopherol, resveratrol, lactic acid and/or ascorbic acid.
Embodiment 16. The method according to any one of embodiments 1 to 15, further comprising grinding after lyophilizing.
Embodiment 17. The method according to any one of embodiments 1 to 16, wherein (a) the sugar or sugar alcohol and/or (b) the amino acid is added upon addition as stabilizer(s).
Embodiment 18. The method according to any of embodiments 1 to 17, wherein the amino acid is present in a pharmaceutically acceptable salt form, in particular before addition, preferably as a hydrochloride, sulfate, acetate, phosphate, tartrate, or citrate salt, in particular arginine hydrochloride, arginine sulfate, arginine acetate, histidine hydrochloride, histidine sulfate, histidine acetate, lysine hydrochloride, lysine sulfate, or lysine acetate.
Embodiment 19. The method according to embodiment 18, wherein the amino acid is arginine and, in particular prior to addition, is present as arginine hydrochloride.
Embodiment 20. A stabilised lyophilisate with plant-based DAO, obtainable by the method according to any one of embodiments 1 to 19.
Embodiment 21. A stabilized lyophilisate with plant-based DAO comprising (a) a sugar or sugar alcohol and (b) a free amino acid.
Embodiment 22. The stabilized lyophilisate according to embodiment 20 or 21, wherein the sugar or sugar alcohol (a) serves as a stabilizer.
Embodiment 23. The stabilized lyophilisate according to any of embodiments 20 to 22, wherein the free amino acid (b) serves as a stabilizer.
Embodiment 24. The stabilized lyophilisate according to embodiment 20 or 23, wherein the sugar or sugar alcohol is present in a weight ratio of at least 1:1 or even at least 2:1, preferably of at least 4:1 or even at least 7:1, preferably at least 10:1 or even at least 13:1, more preferably at least 16:1 or even at least 19:1, in particular at least 22:1 or even at least 25:1 to the total protein content of the lyophilisate.
Embodiment 25. The stabilized lyophilisate according to any one of embodiments 20 to 24, wherein the free amino acid is present in a weight ratio of at least 1:1, preferably at least 4:1 or even at least 7:1, preferably at least 10:1 or even at least 18:1, more preferably at least 25:1 or even at least 32:1, in particular at least 40:1 or even at least 50:1 to the total protein content of the lyophilisate.
Embodiment 26. The stabilized lyophilisate according to any one of embodiments 20 to 25, wherein the free amino acid is present in a pharmaceutically acceptable salt form, preferably as a hydrochloride, sulfate, acetate, phosphate, tartrate or citrate salt, in particular arginine hydrochloride, arginine sulfate, arginine acetate, histidine hydrochloride, histidine sulfate, histidine acetate, lysine hydrochloride, lysine sulfate or lysine acetate.
Embodiment 27. The stabilized lyophilisate of any one of embodiments 20 to 26, wherein the free amino acid is selected from the group consisting of basic amino acids such as arginine, histidine, and lysine, and amino acids asparagine, glutamine, cysteine, homocysteine, methionine, tryptophan, and tyrosine.
Embodiment 28. The stabilized lyophilisate of any of embodiments 20 to 27, wherein the free amino acid is a basic amino acid.
Embodiment 29. The stabilized lyophilisate of any of embodiments 20 to 28, wherein the free amino acid is arginine.
Embodiment 30. The stabilized lyophilisate according to embodiment 29, wherein arginine is present as arginine hydrochloride.
Embodiment 31. The stabilized lyophilisate according any one of embodiments 20 to 30, wherein the sugar or sugar alcohol is selected from the group consisting of disaccharides such as trehalose, sucrose, lactose, and maltose, trisaccharides such as raffinose, polysaccharides such as maltodextrin, and the sugar alcohols mannitol, sorbitol, glycerol, xylitol, erythritol, and inositol.
Embodiment 32. The stabilized lyophilisate according to any of embodiments 20 to 31, wherein the sugar or sugar alcohol is a disaccharide.
Embodiment 33. The stabilized lyophilisate according to any of embodiments 20 to 32, wherein the sugar or sugar alcohol is trehalose.
Embodiment 34. The stabilised lyophilisate according to any of embodiments 20 to 33, wherein the lyophilisate exhibits a DAO activity of at least 1 kHDU/g, preferably of at least kHDU/g (preferably measured with DAO REA); preferably wherein the DAO activity in the lyophilisate is at least 5000 kHDU/g, preferably at least 10000 kHDU/g, even more preferably at least 12500 kHDU/g, in particular at least 15000 kHDU/g or even at least 25000 kHDU/g (preferably measured with DAO REA).
Embodiment 35. A solid, preferably enteric oral dosage form comprising the stabilized lyophilisate according to any one of embodiments 20 to 34, optionally in compressed form.
Embodiment 36. The solid dosage form according to embodiment 35 for therapeutic use, preferably in the treatment of histamine-induced disorders.
Embodiment 37. The use of the solid dosage form according to embodiment 35 as a dietary supplement or dietary food.

### EXAMPLES

The invention is explained in more detail below using preferred, non-limiting examples.

### Example 1—Stability Experiments

Stability experiments were carried out with plant-based DAO and various possible stabilisers both with regard to lyophilisation and subsequent storage. Selected experiments are described below.

Experiments with sucrose or maltodextrin as sole stabilizers did not show sufficient stabilization of the lyophilized DAO from pea sprouts (Pisum sativum) under long-term storage conditions at 25° C. and 60% relative humidity (RH).

In a further experiment, various amino acids (up to 10% w/w) were added to a DAO rich pea sprout homogenate fraction containing 10% w/w maltodextrin. Reduced glutathione has also been added to act as an antioxidant. The homogenate was then lyophilized. After lyophilization, the lyophilisate contained 3-4% (w/w) pea sprout dry matter (mainly pea sprout protein, including the DAO). Long-term storage (up to six months) at 25° C., 60% RH showed that the addition of the amino acids cysteine or methionine, in combination with maltodextrin, led to improved long-term stability of the DAO in the powder of the pea sprout homogenate. Outstanding results (up to 60% residual activity of the DAO after 6 months) were achieved with arginine in combination with the polysaccharide maltodextrin.

Particularly good results were also achieved with histidine in combination with maltodextrin.

In another experiment, trehalose was used as a stabilizer. Using a Design of Experiments (DoE) approach, the use in combination with arginine and maltodextrin was simultaneously tested, and different concentrations of the mixtures were investigated. In order to stabilize the pea sprout homogenate, it was mixed with maltodextrin, trehalose and arginine in different proportions by weight, lyophilized and stored at 30° C., 65% RH in an open container for stability determination. Formulations with arginine and trehalose, but without maltodextrin, provided the best DAO stability values in this experimental approach.

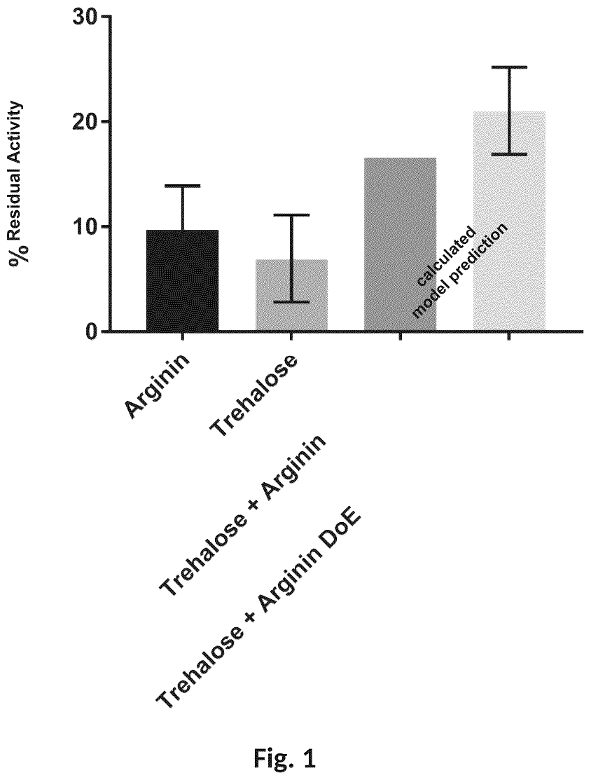

The fact that this was even a more than additive effect could be deduced from the DoE model. If one calculates the sum of the residual activity from the use of trehalose after 6 months of storage at 30° C., 65% RH in an open container of 6.9% and the residual activity from the use of arginine after 6 months of storage at 30° C., 65% RH in an open container of 9.7%, a calculated value of 16.6% residual activity for a combination of trehalose and arginine results. However, the expected value predicted from the DoE model for the combination of trehalose with arginine is 21.1% (with a 95% confidence interval of 16.9% to 25.2%). Consequently, there is a synergistic effect. This is illustrated in FIG. 1.

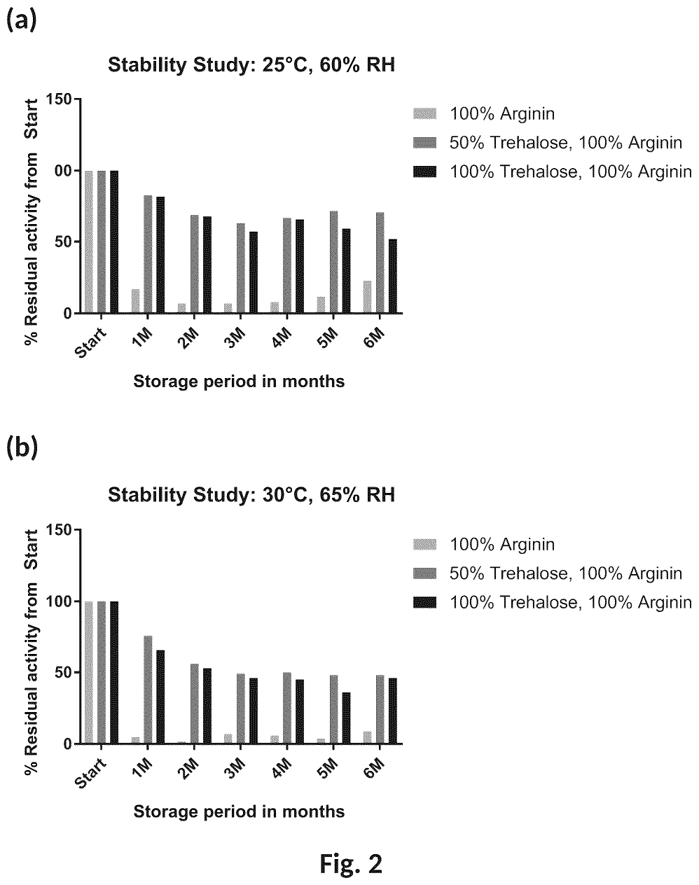

Based on the above results, three formulations were selected, produced and stored for verification. The above results were thus confirmed. In this experiment, a residual activity after 6 months of approx. 60% at 25° C., 60% RH, and of approx. 40% at 30° C., 65% RH was obtained when 1:2 trehalose was chosen as the formulation for pea sprout homogenate and 1:1 arginine (in the form of the monohydrochloride of arginine) for pea sprout homogenate or 1:1 trehalose for pea sprout homogenate and 1:1 arginine (in the form of the monohydrochloride of arginine) for pea sprout homogenate (data on a weight basis). If, in comparison, only 1:1 arginine (in the form of the monohydrochloride of arginine) was added to the pea sprout homogenate, the activity of the DAO had already fallen to below 20% of the starting activity after one month. (see FIGS. 2a and b.)

### Example 2—Preparation of the Dosage Form According to the Invention

Pea sprouts (Pisum sativum) containing DAO were provided. To produce the pea sprout homogenate, the germinated peas were homogenized with a first part of water in a blender.

The homogenate had a DAO activity of approx. 90,000 kHDU/g. Subsequently, the homogenate was diluted to improve the pipettability. To separate fiber parts, the homogenate was centrifuged at 400×g for 15 minutes. The supernatant was centrifuged again at 11,000 xg for 1 h 50 min and the supernatant was sterile-filtered. This sterile-filtered supernatant was the fraction of the homogenate intended for lyophilization. The determination of the DAO activity in the respective processing steps showed that the centrifugation and filtration steps did not require any loss of activity.

Subsequently, the resulting pea sprout homogenate was mixed with one part arginine (100% w/w arginine monohydrochloride to pea sprout homogenate) and one half part trehalose (50% w/w trehalose to pea sprout homogenate). This resulted in a mixture (on a weight basis) of ⅖ pea sprout homogenate, ⅖ arginine monohydrochloride and ⅕ trehalose (the mixing ratio was normalized to the total protein content, on a weight basis:1 part total protein:25 parts trehalose:50 parts arginine hydrochloride). This mixture was frozen and then lyophilized and ground. After lyophilization, the DAO activity of the powder was converted to approx. 60,000 kHDU/g lyophilisate, or, calculated back to the pea sprout homogenate, to approx. 90,000 kHDU/g homogenate. (In one test batch, powder obtained after lyophilization was stored in closed containers at 25° C. and 60% RH for 5 months, the DAO activity was still above 60,000 kHDU/g homogenate after conversion.)

For the preparation of the dosage form according to the invention, the powder is mixed with the following additives after lyophilization: Microcrystalline cellulose, silicon dioxide, cross-carmellose sodium, magnesium strearate and tabletted. In order to prevent inactivation of the enzyme by the gastric passage, an enteric coating consisting of shellac is applied.

### Example 3—Synergistic Effect of the Additives

In order to shed more light on the synergistic effect of trehalose and arginine as lyophilization and storage stabilizers characterized in Example 1, further stability tests were carried out with plant-based DAO.

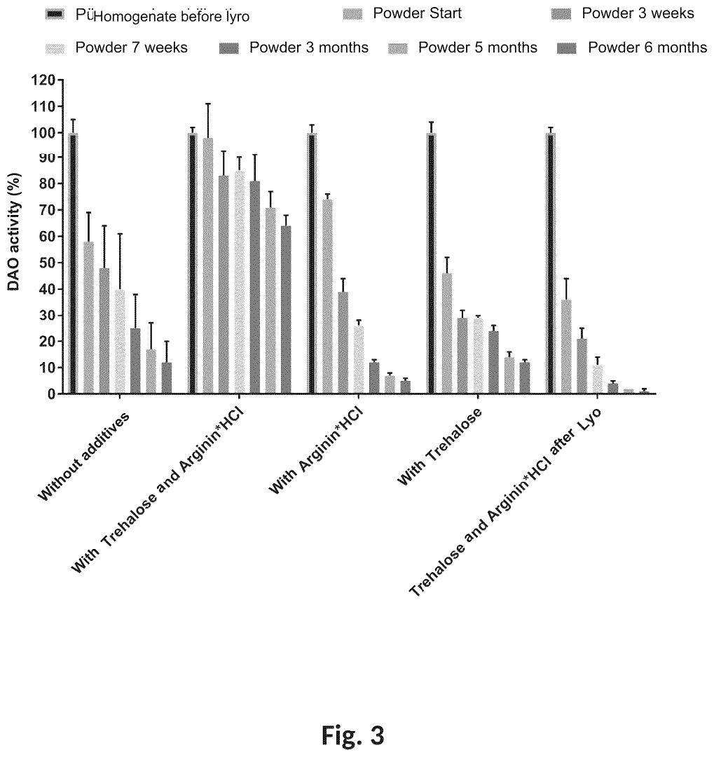

For this purpose, DAO-rich pea sprout homogenate (DAO activity: 92,000 kHDU/g homogenate) was prepared. The pea sprout homogenate was lyophilized without additives or lyophilized with trehalose and arginine hydrochloride in the ratio of 1 part total protein:25 parts trehalose:50 parts arginine hydrochloride (on a weight basis) or only with arginine hydrochloride (50 parts: 1 part total protein (on a weight basis)) or only with trehalose (25 parts:1 part total protein (on a weight basis)). FIG. 3 shows that the joint addition of trehalose and arginine hydrochloride leads to a significantly better stabilizing effect on the DAO during lyophilization (98% residual activity powder after lyophilization) than without additives (58% residual activity powder after lyophilization) or only with individual components (74% residual activity powder after lyophilization with only arginine hydrochloride or 46% residual activity after lyophilization with only trehalose).

As an additional experiment, lyophilized pea sprout homogenate without additives was mixed with trehalose and arginine hydrochloride in a ratio of 1 part total protein:25 parts trehalose:50 parts arginine hydrochloride (on a weight basis) only after lyophilization.

The powders obtained after lyophilization were then stored in closed containers at 25° C. and 60% RH for 6 months.

The 6-month values clearly showed that the DAO enzyme activity of the lyophilized pea sprout homogenate without additives decreased sharply (12% residual activity compared to before lyophilization), that the addition of arginine hydrochloride or trehalose alone did not bring any improvement, and that the addition of these stabilizers did not bring any stabilization in this experiment until after lyophilization (only 1% residual activity compared to before lyophilization).

In contrast, the combined addition of arginine hydrochloride and trehalose prior to lyophilization resulted in a substantial retention of DAO enzyme activity, both during lyophilization and during storage at 25° C. and 60% RH. This was due to a synergistic effect by combining both additives: The combined application of the two additives resulted in a significantly higher residual activity (64% residual activity compared to before lyophilization) than the stabilizing effect of the individual substances added (only arginine 5%+only trehalose 12%=added 17% residual activity compared to before lyophilization).

### Example 4—Addition of Additives in Different Amounts Relative to the Total Protein

In further stability studies, it was investigated how additions of the additives in different amounts had an effect. On the one hand, 1 part of total protein was mixed with 12.5 parts of trehalose and 25 parts of arginine hydrochloride (on a weight basis) and, on the other hand, 1 part of total protein was mixed with twice the amount of additives, i.e. 25 parts of trehalose and 50 parts of arginine hydrochloride (on a weight basis), and both variants were lyophilized. The powders obtained were stored in closed containers at 25° C. and 60% RH and tested for DAO enzyme activity for 24 months.

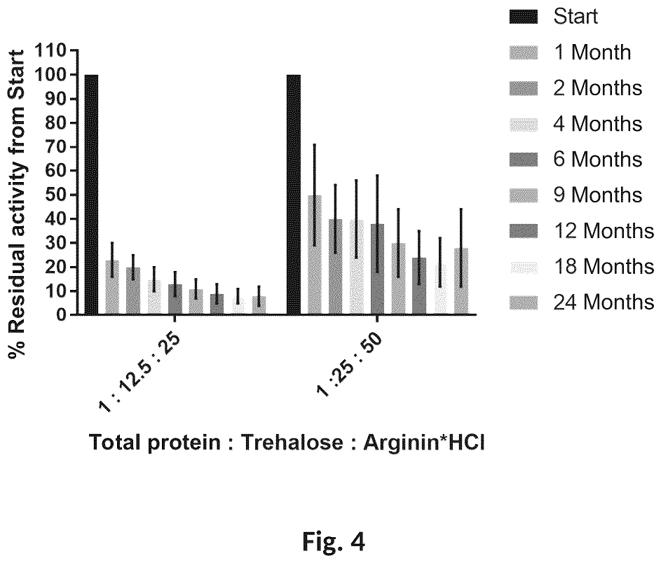

It can be seen from FIG. 4 that the doubling of the amount of additive investigated here led to an improvement in the long-term DAO stability.

1-22. (canceled)

23. A method for producing a stabilized lyophilisate with plant-based diamine oxidase (DAO), comprising the following steps:
providing DAO-containing plants or DAO-containing parts thereof;
homogenizing the DAO-containing plants or DAO-containing parts thereof in an aqueous solution to obtain an aqueous homogenate;
adding (a) a sugar or sugar alcohol and (b) an amino acid; and
lyophilizing the aqueous homogenate or a DAO-containing fraction thereof in the presence of (a) the sugar or sugar alcohol and (b) the amino acid to obtain the stabilized lyophilisate.

24. The method of claim 23, wherein the amino acid is selected from the group consisting of basic amino acids and the amino acids asparagine, glutamine, cysteine, homocysteine, methionine, tryptophan, and tyrosine.

25. The method of claim 24, wherein the amino acid is a basic amino acid.

26. The method of claim 25, wherein the amino acid is selected from the group consisting of arginine, histidine and lysine.

27. The method of claim 25, wherein the amino acid is arginine.

28. The method of claim 23, wherein the amino acid is present in a pharmaceutically acceptable salt form.

29. The method of claim 28, wherein the amino acid is present as a hydrochloride, sulfate, acetate, phosphate, tartrate or citrate salt.

30. The method of claim 27, wherein the amino acid is arginine and is present as arginine hydrochloride.

31. The method of claim 23, wherein the sugar or sugar alcohol is selected from the group consisting of disaccharides, trisaccharides, polysaccharides, and the sugar alcohols mannitol, sorbitol, glycerol, xylitol, erythritol, and inositol.

32. The method of claim 31, wherein the sugar or sugar alcohol is a disaccharide.

33. The method of claim 32, wherein the sugar or sugar alcohol is trehalose.

34. The method of claim 23, wherein the DAO-containing plants are members of the Leguminosae family.

35. The method of claim 34, wherein the DAO-containing plants are selected from the genera Pisum, Lens, Cicer, Lathyrus, Phaseolus, and Vicia.

36. The method of claim 23, wherein the sugar or sugar alcohol is present in a weight ratio of at least 1:1 to the total protein content of the stabilized lyophilisate.

37. The method of claim 36, wherein the sugar or sugar alcohol is present in a weight ratio of at least 2:1 to the total protein content of the stabilized lyophilisate.

38. The method of claim 23, wherein the amino acid is present in a weight ratio of at least 1:1 to the total protein content of the stabilized lyophilisate.

39. The method of claim 23, wherein the stabilized lyophilisate exhibits a DAO-activity of at least 1 kHDU/g.

40. The method of claim 39, wherein the stabilized lyophilisate exhibits a DAO-activity of at least 5000 kHDU/g.

41. The method of claim 23, further comprising providing a solid oral dosage form comprising the stabilized lyophilisate and optionally further excipients, optionally in compressed form.

42. A method for producing a solid oral dosage form of plant-based DAO, comprising the following steps:
providing DAO-containing plants or DAO-containing parts thereof;
homogenizing the DAO-containing plants or DAO-containing parts thereof in an aqueous solution to obtain an aqueous homogenate;
adding (a) a sugar or sugar alcohol and (b) an amino acid;
lyophilizing the aqueous homogenate or a DAO-containing fraction thereof in the presence of (a) the sugar or sugar alcohol and (b) the amino acid to obtain a stabilized lyophilisate, wherein the stabilized lyophilisate exhibits a DAO-activity of at least 10 kHDU/g, wherein the sugar or sugar alcohol is present in a weight ratio of at least 2:1 to the total protein content of the stabilized lyophilisate, wherein the amino acid is present in a weight ratio of at least 1:1 to the total protein content of the stabilized lyophilisate;
optionally grinding and/or compressing the stabilized lyophilisate;
adding one or more further excipients to the stabilized lyophilizate; and
providing a solid oral dosage form comprising the, optionally ground and/or compressed, stabilized lyophilisate and one or more further excipients, wherein the solid oral dosage form has an enteric coating.

43. A method for producing a solid oral dosage form of plant-based DAO, comprising the following steps:
providing DAO-containing plants or DAO-containing parts thereof, wherein the DAO-containing plants are members of the Leguminosae family;
homogenizing the DAO-containing plants or DAO-containing parts thereof in an aqueous solution to obtain an aqueous homogenate;
adding (a) a sugar or sugar alcohol which is a disaccharide or trisaccharide and (b) an amino acid which is arginine or a pharmaceutically acceptable salt thereof;
lyophilizing the aqueous homogenate or a DAO-containing fraction thereof in the presence of (a) the sugar or sugar alcohol and (b) the amino acid to obtain a stabilized lyophilisate, wherein the stabilized lyophilisate exhibits a DAO-activity of at least 10 kHDU/g, wherein the sugar or sugar alcohol is present in a weight ratio of at least 2:1 to the total protein content of the stabilized lyophilisate, wherein the amino acid is present in a weight ratio of at least 1:1 to the total protein content of the stabilized lyophilisate;
grinding the stabilized lyophilisate;
adding one or more further excipients to the ground stabilized lyophilisate;
optionally compressing the ground stabilized lyophilisate with the excipients; and
providing a solid oral dosage form comprising the ground, optionally compressed, stabilized lyophilisate, wherein the solid oral dosage form has an enteric coating.
